(19) United States
(12) Patent Application Publication
(10) Pub. No.: US 2022/0257192 A1
(43) Pub. Date: Aug. 18, 2022

(54) DEVICE AND METHOD OF MANUFACTURING THE SAME

(71) Applicant: NEC Cosporation, Minato-ku, Tokyo (JP)

(72) Inventors: Ni Ni SOE, Singapore (SG); Charles CHOY, Singapore (SG)

(73) Assignee: NEC Cosporation, Minato-ku, Tokyo (JP)

(21) Appl. No.: 17/623,722

(22) PCT Filed: Jun. 26, 2020
(86) PCT No.: PCT/JP2020/025172

§ 371 Date: Dec. 29, 2021

(30) Foreign Application Priority Data
Jul. 10, 2019 (SG) 10201906405P

(51) Int. Cl. A61B 5/00; A61B 5/296

(52) U.S. Cl. CPC A61B 5/6841; A61B 2562/12; A61B 5/6835; A61B 5/296

(57) ABSTRACT

A device and a method of manufacturing the device may be provided. The device (10) includes a support member (11); two or more electrodes (12) configured to obtain data, the two or more electrodes (12) being positioned on the support member (11); and one or more location identifiers (13) positioned on the support member (11), each of the one or more location identifiers (13) identifying a location to position one of the two or more electrodes (12) to obtain data corresponding to the one or more muscle groups of a body.

### TECHNICAL FIELD

The present disclosure relates to a device and a method for manufacturing the device.

### BACKGROUND ART

Brain injuries, such as stroke or traumatic brain injury, are the leading cause in the world of death and also contribute significantly to patients' disability. Brain injury patients require assistance or they may become fully dependent on their caregivers for their Activities of Daily Living (ADL), which may require neuromuscular monitoring and treatment.

An example of neuromuscular monitoring is Electromyography (EMG), in which EMG sensors with electrodes are used in a clinical environment to collect electrical activity of the muscles in order to measure the strength of a specific muscle group. EMG measurement is important to monitor patients' functional recovery, such as stroke rehabilitation.

### SUMMARY OF INVENTION

### Technical Problem

The electrodes of the EMG sensors are positioned manually on the muscles by clinicians to collect the muscle activity, via electrical signals, from the specific muscle group. Therefore, clinicians have to position the electrode in the correct muscle group to monitor specific activity of patient.

EMG is typically conducted by experienced personnel (for example clinicians) in a clinic or laboratory, because it is difficult to consistently place the electrodes at the same location of a single individual patient across multiple time points. If the electrodes are placed at the wrong location or at a different muscle group, crosstalk of the electrical signals may occur. Inconsistent placement of the electrodes may also raise difficulties in obtaining accurate and noise-free data.

An object of the present disclosure is to provide a device and a method capable of obtaining data of one or more muscle groups of a body for rehabilitation.

### Solution to Problem

In a first example aspect, a device comprises: a support member; two or more electrodes configured to obtain data, the two or more electrodes being positioned on the support member; and one or more location identifiers positioned on the support member, each of the one or more location identifiers identifying a location to position one of the two or more electrodes to obtain data corresponding to one or more muscle groups of a body.

In a second example aspect, a method of manufacturing a device comprises: providing a support member; positioning two or more electrodes on the support member, the two or more electrodes configured to obtain data; and positioning one or more location identifiers on the support member, each of the one or more location identifiers identifying a location to position one of the two or more electrodes to obtain data corresponding to one or more muscle groups of a body.

### Advantageous Effects of Invention

According to the present disclosure, it is possible to provide a device and a method capable of obtaining data of one or more muscle groups of a body for rehabilitation.

### BRIEF DESCRIPTION OF DRAWINGS

FIG. 1 shows a top perspective view of a device for obtaining data of one or more muscle groups of a body for rehabilitation according to a first example embodiment.

FIG. 2A shows a top perspective view of the device of FIG. 1 illustrating muscle groups of an upper limb in which data is obtained by the two or more electrodes according to the first example embodiment.

FIG. 2B shows a top perspective view of the device of FIG. 1 illustrating locations of the two or more location identifiers according to the first example embodiment.

FIG. 3A shows a schematic diagram illustrating a top perspective view of the device of FIG. 1 when used on an arm of a user according to the first example embodiment.

FIG. 3B shows a schematic diagram illustrating a top perspective view of the device of FIG. 1 on the arm of the user with two or more disposable electrodes attached thereto according to the first example embodiment.

FIG. 3C shows a schematic diagram illustrating a bottom perspective view of the device of FIG. 1 with two or more disposable electrodes attached thereto according to the first example embodiment.

FIG. 3D shows a schematic diagram illustrating a top perspective view of the device of FIG. 1 on the arm of the user with two or more disposable electrodes each having a snap cable attached thereto according to the first example embodiment.

FIG. 3E shows a schematic diagram illustrating a top perspective view of the device of FIG. 1 on the arm of the user with two or more disposable electrodes connected to a wireless EMG sensor according to the first example embodiment.

FIG. 4A shows a top perspective view of the device of FIG. 1 for use on a left arm of the user according to the first example embodiment.

FIG. 4B shows a top perspective view of the device of FIG. 1 for use on a right arm of the user according to the first example embodiment.

FIG. 5 shows a schematic diagram illustrating a top perspective view of the device of FIGS. 4A and 4B secured on the left arm and right arm of the user respectively according to the first example embodiment.

FIG. 6 shows a perspective view illustrating dimensions of a forearm of a user for which the device of FIG. 1 is to be used in a study according to the first example embodiment.

FIG. 7A shows graphs illustrating forearm muscle group data of various subjects in the study using the device of FIG. 1, according to the first example embodiment.

FIG. 7B shows graphs illustrating forearm muscle group data of various subjects in the study using the device of FIG. 1, according to the first example embodiment.

FIG. 7C shows graphs illustrating forearm muscle group data of various subjects in the study using the device of FIG. 1 according to the first example embodiment.

FIG. 8 shows a top perspective view of a device for obtaining data of one or more muscle groups of a body for rehabilitation according to the first example embodiment.

FIG. 9A shows a top perspective view of the device of FIG. 8 illustrating muscle groups of a lower limb in which data is obtained by the two or more electrodes according to the first example embodiment.

FIG. 9B shows a top perspective view of the device of FIG. 8 illustrating locations of the one or more location identifiers according to the first example embodiment.

FIG. 10A shows a schematic diagram illustrating a top perspective view of the device of FIG. 8 when used on a leg of a user according to the first example embodiment.

FIG. 10B shows a schematic diagram illustrating a top perspective view of the device of FIG. 8 on the leg of the user with two or more disposable electrodes attached thereto according to the first example embodiment.

FIG. 10C shows a schematic diagram illustrating a bottom perspective view of the device of FIG. 8 with two or more disposable electrodes attached thereto according to the first example embodiment.

FIG. 10D shows a schematic diagram illustrating a top perspective view of the device of FIG. 8 on the leg of the user with two or more disposable electrodes each having a snap cable attached thereto according to the first example embodiment.

FIG. 10E shows a schematic diagram illustrating a top perspective view of the device of FIG. 8 on the leg of the user with two or more disposable electrodes connected to a wireless EMG sensor according to the first example embodiment.

FIG. 11A shows a top perspective view of the device of FIG. 8 for use on a right leg of the user according to the first example embodiment.

FIG. 11B shows a top perspective view of the device of FIG. 8 for use on a left leg of the user according to the first example embodiment.

FIG. 12 shows a schematic diagram illustrating a top perspective view of the device of FIGS. 11A and 11B secured on the left leg and right leg of the user respectively according to the first example embodiment.

FIG. 13 shows a flow chart illustrating a method for obtaining data of one or more muscle groups of a body for rehabilitation according to the first example embodiment.

FIG. 14 shows a block diagram illustrating the structure of a device according to the second example embodiment.

### DESCRIPTION OF EMBODIMENTS

Prior to explaining embodiments according to this present disclosure, the following explanatory notes will be given.

As shown above, EMG is typically conducted by experienced personnel (for example clinicians) in a clinic or laboratory. The first reason for this is that it is difficult to consistently place the electrodes at the same location of a single individual patient across multiple time points. The second reason for this is that it is difficult to find a correct location of electrodes on muscles in different patients when placing the electrodes manually. Another reason is that if EMG is not conducted by such experienced personnel, loss of contact between the skin and the electrodes may occur, thereby resulting in the incomplete recording of the electrical (or EMG) signals. Due to these above reasons, EMG is therefore seldom performed by inexperienced personnel outside of a clinic or laboratory.

Currently, devices which stimulate muscles for rehabilitation or pain relief while monitoring systems or rehabilitation devices using EMG can only be used by experienced personnel in a clinical environment. There is thus a need to provide a device that can be easily removed from a patient, and that is washable and wearable so that it can be used for EMG activity while using conventional electrodes by non-experienced personnel.

There is also a need to have a device that can provide reliable, accurate and correct positioning of electrodes consistently for prolonged periods of recording in EMG in order to monitor the progress of a patient's rehabilitation.

Herein disclosed are embodiments of a device and methods for obtaining data of one or more muscle groups of a body for rehabilitation that addresses one or more of the above problems.

Furthermore, other desirable features and characteristics will become apparent from the subsequent detailed description and the appended claims, taken in conjunction with the accompanying drawings and the background of the disclosure.

The accompanying Figures, where like reference numerals refer to identical or functionally similar elements throughout the separate views and which together with the detailed description below are incorporated in and form part of the specification, serve to illustrate various embodiments and to explain various principles and advantages in accordance with a present embodiment, by way of non-limiting example only.

Embodiments of this disclosure will be better understood and readily apparent to one of ordinary skill in the art from the following written description, by way of example only, and in conjunction with the drawings.

### First Example Embodiment

A first example embodiment of the disclosure is explained below referring to the accompanying drawings. The following detailed description is merely exemplary in nature and is not intended to limit this disclosure or the application and uses of this disclosure. Furthermore, there is no intention to be bound by any theory presented in the preceding background of this disclosure or the following detailed description. Herein, a device for obtaining data of one or more muscle groups of a body for rehabilitation are presented in accordance with present embodiments having the advantages of providing reliable and accurate data of muscles for rehabilitation as well as correct positioning of electrodes.

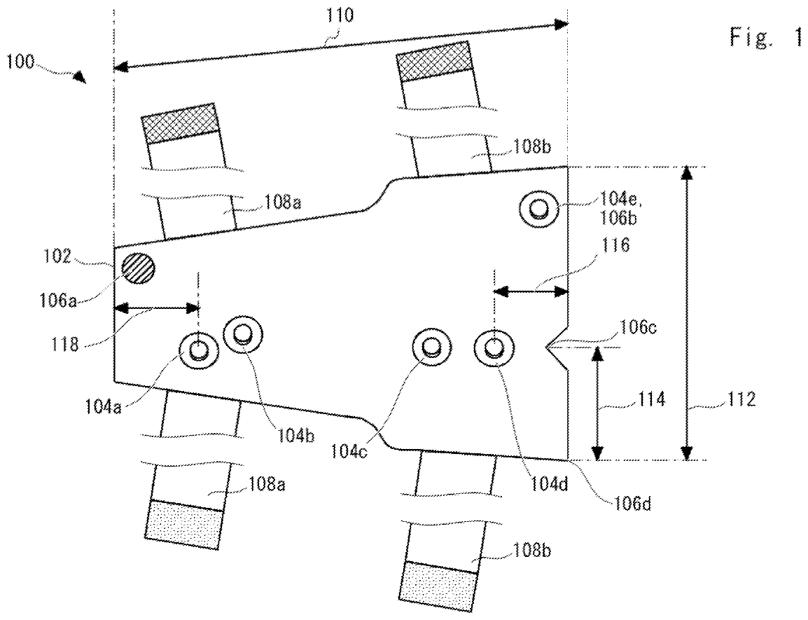

FIG. 1 shows a top perspective view of a device 100 for obtaining data of one or more muscle groups of a body for rehabilitation according to an example embodiment. The device 100 includes a support member 102, two or more electrodes 104a, 104b, 104c, 104d, 104e positioned on the support member 102 and two or more location identifiers 106a, 106b, 106c, 106d that are also positioned on the support member 102.

The support member 102 may be elastic such that it can be stretched to adapt to different sizes of the body of a user. Being stretchable, the support member 102 may be adapted to circumferentially enclose a portion of the body relating to the one or more muscle groups regardless of the body size. In addition, the support member 102 may be made from a soft, non-rigid material such as polyester or vinyl. The support member 102 may be dimensioned approximately 21 centimeters in length 110 and approximately 15 centimeters in width 112.

The two or more electrodes 104a, 104b, 104c, 104d, 104e may be configured to obtain data corresponding to the one or more muscle groups while the two or more location identifiers 106a, 106b, 106c, 106d can be permanently fixed or removably attached to the support member 102. Even though five electrodes and four location identifiers are shown in FIG. 1, it can be appreciated that other embodiments of the device 100 may include more than five electrodes and more than four location identifiers.

Each of the two or more electrodes 104a, 104b, 104c, 104d, 104e may be a disposable electrode such as a “peel-and-stick” electrode typically used in electrocardiogram. Other types of electrodes, such as dry electrodes, surface electrodes, gelled electrodes, needle electrodes and/or fine wire electrodes are also possible. The two or more electrodes 104a, 104b, 104c, 104d, 104e may obtain data from the one or more muscle groups to detect medical abnormalities, activation level of the muscle, or to analyse the biomechanics of the user.

In the embodiment where the two or more electrodes 104a, 104b, 104c, 104d, 104e may be removable from the support member 102, the device 100 may include two or more electrode mounts configured to receive the two or more electrodes 104a, 104b, 104c, 104d, 104e respectively. The function of the electrode mounts is to better secure the electrodes. In an embodiment, the two or more electrodes 104a, 104b, 104c, 104d, 104e may obtain electrical signals from the one or more muscle groups to assist in muscle activity. In an alternate embodiment, the two or more electrodes 104a, 104b, 104c, 104d, 104e may be configured to transmit data to the one or more muscle groups of the body. For example, the two or more electrodes 104a, 104b, 104c, 104d, 104e may transmit electrical signals to the one or more muscle groups to assist in muscle stimulation. The transmission of electrical signals for muscle stimulation may assist to relieve pain for the one or more muscle groups.

Each of the two or more electrodes 104a, 104b, 104c, 104d, 104e may be located at a predetermined length relative to an edge of the support member 102. For example, as shown in FIG. 1, electrode 104a may be at a length of approximately 3.5 centimeters to a first edge of the support member 102, starting at reference numeral 118 (measured from the center of the electrode) to the first edge. Electrode 104b may thus be at a relative distance of approximately 2 centimeters (measured from the center of the electrode) to electrode 104a. Similarly, electrode 104d may be at a length of approximately 5 centimeters, starting at reference numeral 116 (measured from the center of the electrode) to a second edge that is opposite the first edge of the support member 102. Electrode 104c may thus be at a relative distance of approximately 3.5 centimeters (measured from the center of the electrode) to electrode 104d.

Each of the two or more location identifiers 106a, 106b, 106c, 106d may identify a location to position one of the two or more electrodes 104a, 104b, 104c, 104d, 104e to obtain data corresponding to the one or more muscle groups of the body. Each of the two or more location identifiers 106a, 106b, 106c, 106d may be located at a predetermined length relative to an edge of the support member 102. For example, as shown in FIG. 1, location identifier 106c may be at a length of approximately 5.5 centimeters, starting at reference numeral 114 to an edge of the support member 102 and location identifier 106b may be in the same position with electrode 104e. Further, location identifier 106d may be positioned at a corner of the support member 102.

It can be appreciated that the dimensions of the location identifiers may vary depending on the muscle group of the body. For example, location identifier 106c may be positioned at a different length from the edge of the support member 102 for obtaining data from a muscle group of an upper arm.

The device 100 may also include fastening means 108a, 108b so that the support member 102 can be removably secured against the body. The fastening means may include but not limited to: a hook and loop fastener (e.g. a Velcro ((R): Registered trademark) fastener), a buckle fastener and/or an adhesive fastener. It can be appreciated that other fastening means may be applicable such that the device 100 can be secured and removed from the body with minimal effort.

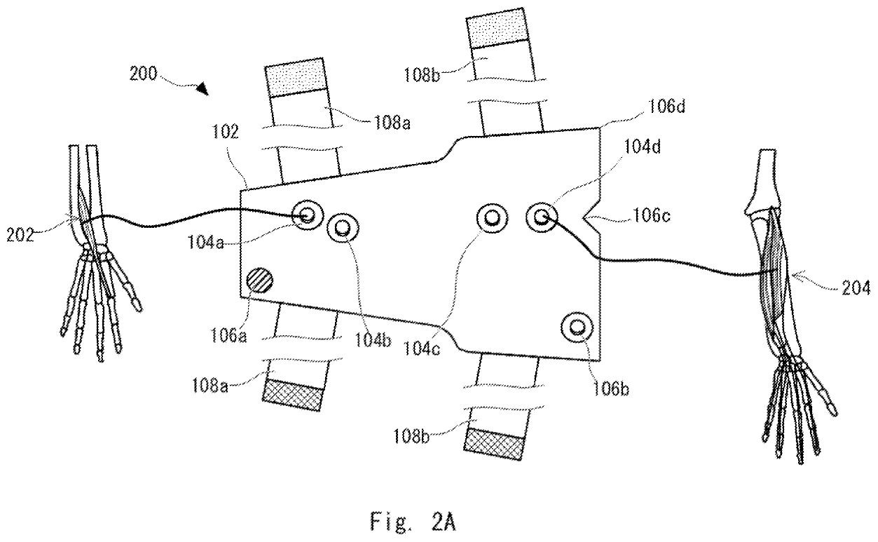

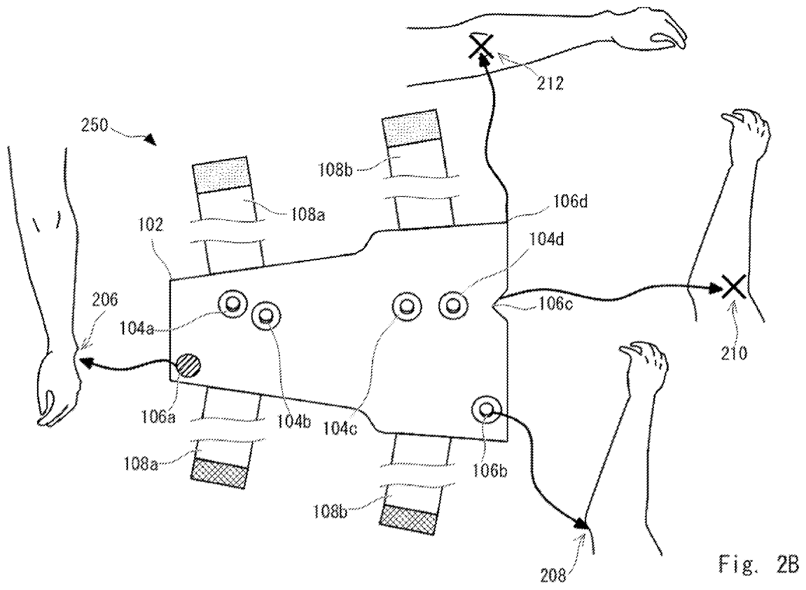

FIG. 2A shows a top perspective view 200 of the device 100 of FIG. 1 illustrating muscle groups of an upper limb in which data is obtained by the two or more electrodes according to an example embodiment. In this embodiment, the one or more muscle groups of the body may refer to the muscle groups of an upper limb, such as an arm of the user. For example and as shown in FIG. 2A, electrodes 104a and 104b may be positioned to obtain data corresponding to the extensor indicis muscle group 202 that is located in the forearm of the user while electrodes 104c and 104d may be positioned to obtain data corresponding to the extensor digitorum muscle group 204 that is also located in the forearm of the user.

FIG. 2B shows a top perspective view 250 of the device 100 of FIG. 1 illustrating locations of the two or more location identifiers 106a, 106b, 106c, 106d according to an example embodiment. In this embodiment, the two or more location identifiers 106a, 106b, 106c, 106d may identify locations of an upper limb, i.e. an arm of the user. For example, and as shown in FIG. 2B, location identifier 106a may identify the location of the Ulnar Styloid 206 which is at a distal end of the forearm while location identifier 106b may identify the location of the olecranon 208, which is the “pointy” part of the elbow. Further, location identifier 106c may identify the location of the elbow axis 210 while location identifier 106d may identify the location of the middle of the elbow joint width 212. When the device 100 is used on the arm of the user, the two or more location identifiers 106a, 106b, 106c, 106d identify the respective locations such that one of the two or more electrodes 104a, 104b, 104c, 104d, 104e are positioned to obtain data corresponding to the one or more muscle groups of the arm.

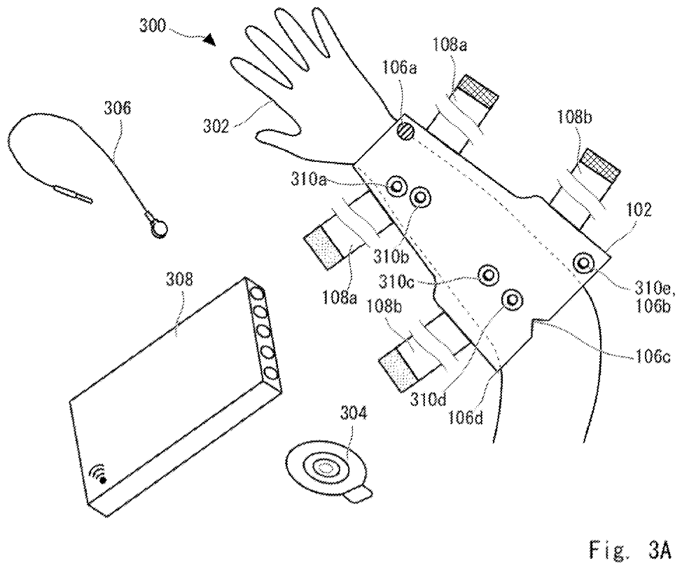

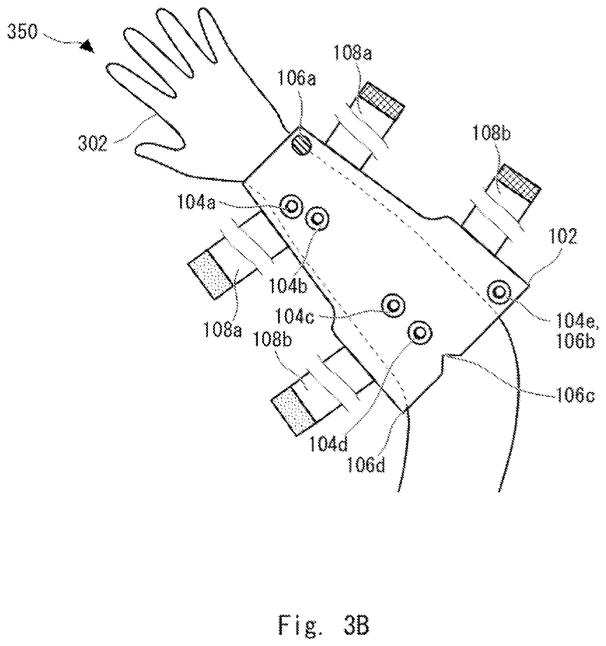

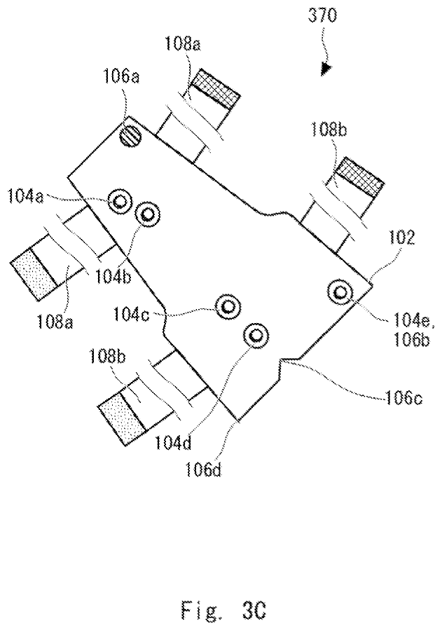

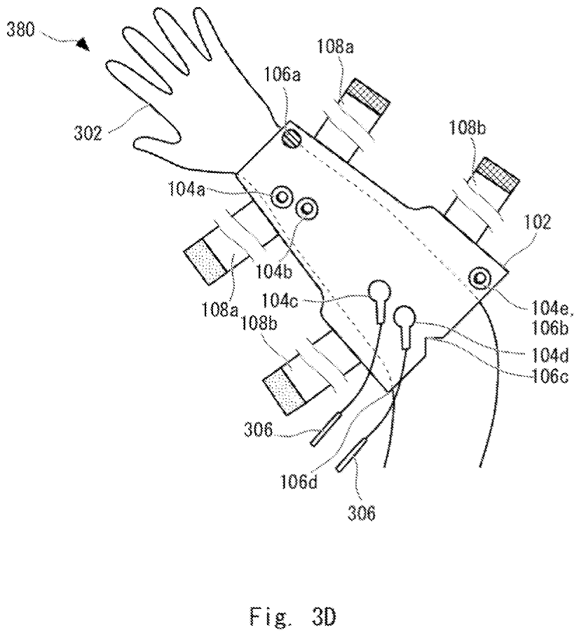

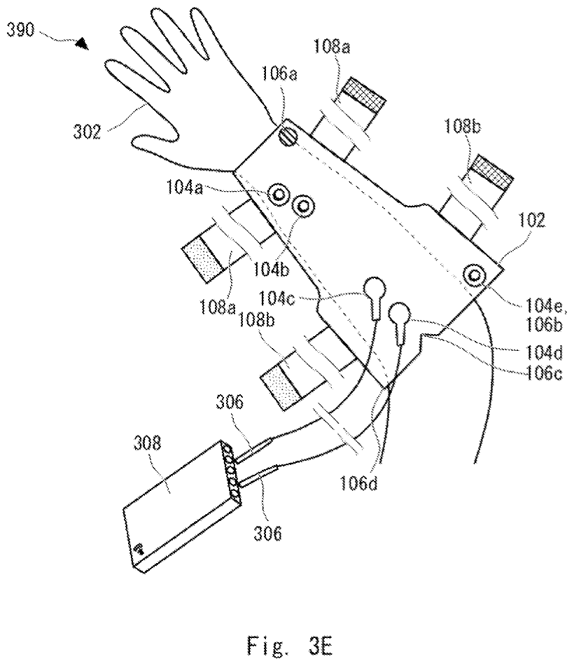

FIG. 3A shows a schematic diagram 300 illustrating a top perspective view of the device 100 of FIG. 1 when used on an arm 302 of a user according to an example embodiment. Before data is collected from the muscle groups, the device 100 is placed on the arm 302 such that the support member 102 circumferentially encloses the arm 302. The user may then adjust the device 100 such that the two or more location identifiers 106a, 106b, 106c, 106d are positioned at the respective locations as described previously. That is, location identifier 106a is positioned at the Ulnar Styloid 206, location identifier 106b is positioned at the location of the olecranon 208, location identifier 106c is positioned at the location of the elbow axis 210 and location identifier 106d is positioned at the location of the middle of the elbow joint width 212. In this way, electrodes 104a and 104b are positioned to obtain data corresponding to the extensor indicis muscle group 202 and electrodes 104c and 104d are positioned to obtain data corresponding to the extensor digitorum muscle group 204. The device 100 is then secured to the arm 302 by using the fastening means 108a, 108b.

In addition, each of the two or more electrodes 104a, 104b, 104c, 104d, 104e may be a disposable electrode 304 that can be readily removed and attached to the device 100. In this embodiment, the support member 102 includes two or more electrode mounts 310a, 310b, 310c, 310d, 310e configured to receive the two or more electrodes 104a, 104b, 104c, 104d, 104e respectively. The disposable electrode 304 may be a typical “peel-and-stick” electrode that is only used once for hygiene purposes. The disposable electrode 304 may also be attached to a snap cable 306 to allow transmission of data to a wireless EMG sensor 308. It can be appreciated that other types of electrodes, such as dry electrodes, surface electrodes, gelled electrodes, needle electrodes and/or fine wire electrodes are possible for each of the two or more electrodes 104a, 104b, 104c, 104d, 104e.

FIG. 3B shows a schematic diagram 350 illustrating a top perspective view of the device 100 of FIG. 1 on the arm 302 of the user with two or more disposable electrodes 104a, 104b, 104c, 104d, 104e attached thereto according to an example embodiment. After the device 100 is secured to the arm 302, the two or more disposable electrodes 104a, 104b, 104c, 104d, 104e are attached to the device 100 via the two or more electrode mounts 310a, 310b, 310c, 310d, 310e. Alternatively, the two or more disposable electrodes 104a, 104b, 104c, 104d, 104e may be attached to the device 100 before being secured to the arm 302. In yet another alternative embodiment, the two or more disposable electrodes 104a, 104b, 104c, 104d, 104e may be permanently attached to the device 100 and therefore the device 100 is secured to the arm 302 without the step of attaching the two or more disposable electrodes 104a, 104b, 104c, 104d, 104e. FIG. 3C shows a schematic diagram 370 illustrating a bottom perspective view of the device 100 of FIG. 1 with two or more disposable electrodes 104a, 104b, 104c, 104d, 104e attached thereto according to an example embodiment. The functions of 104a, 104b, 104c, 104d, 104e in the bottom perspective view may have the same functions as illustrated in FIG. 3B.

FIG. 3D shows a schematic diagram 380 illustrating a top perspective view of the device 100 of FIG. 1 on the arm 302 of the user with two or more disposable electrodes 104a, 104b, 104c, 104d, 104e each having a snap cable 306 attached thereto according to an example embodiment. After the two or more disposable electrodes 104a, 104b, 104c, 104d, 104e are mounted to the device 100, a snap cable 306 may be attached to each of the two or more disposable electrodes 104a, 104b, 104c, 104d, 104e. As shown in FIG. 3D, electrodes 104c and 104d are each attached to a snap cable 306 to facilitate transmission of data of the muscle groups. Each snap cable 306 may be flexible with a “snap-on” feature that can be readily and securely attached to the electrode which may provide strength and stability during the transmission of data.

FIG. 3E shows a schematic diagram 390 illustrating a top perspective view of the device 100 of FIG. 1 on the arm 302 of the user with two or more disposable electrodes 104a, 104b, 104c, 104d, 104e connected to a wireless EMG sensor 308 according to an example embodiment. After attaching the snap cable 306 to each of the two or more disposable electrodes 104a, 104b, 104c, 104d, 104e, each of the snap cable 306 is subsequently connected to the wireless EMG sensor 308. The wireless EMG sensor 308 may be one that measures and records activity of the muscle groups from the data transmitted by the two or more disposable electrodes 104a, 104b, 104c, 104d, 104e. The wireless EMG sensor 308 may be a portable sensor that can be worn by the user and can be used to analyze and assess the biomechanics of the user through muscle contractions, nerve conduction, muscular response in injured tissue and/or activation level. The data received by the wireless EMG sensor 308 can also be transmitted wirelessly to a computing device for further analysis using a suitable software program. In FIG. 3E, the wireless EMG sensor 308 is configured to receive data from at least two of the five electrodes, wherein one of the electrodes provides one channel of EMG data. It can be appreciated that the wireless EMG sensor 308 may be configured to receive data from more than five electrodes.

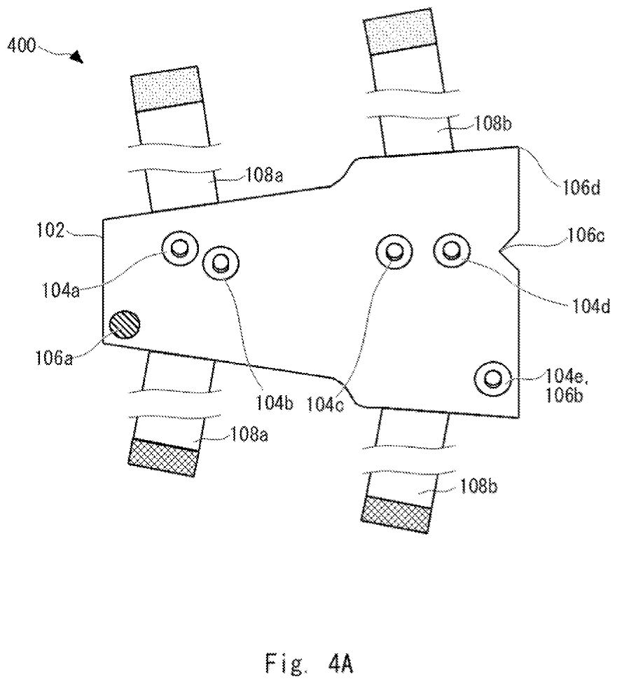

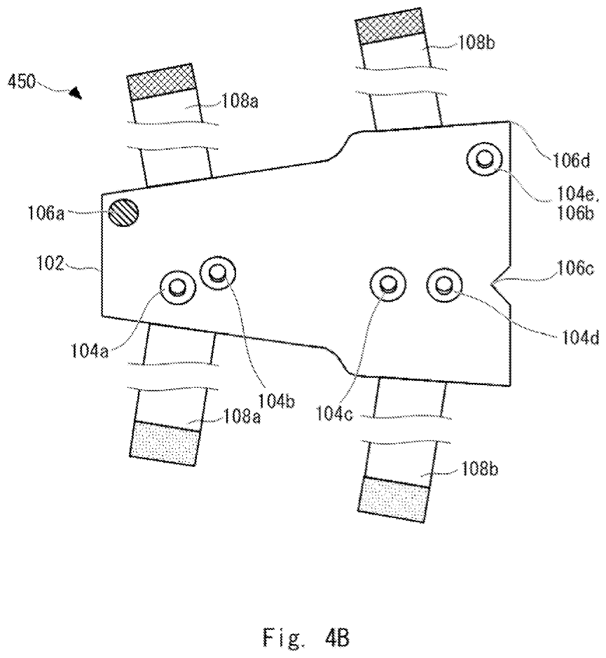

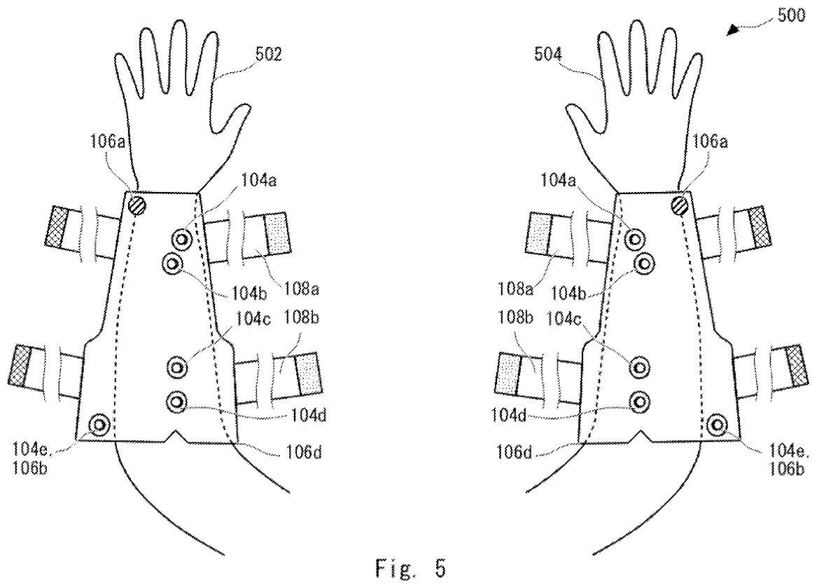

FIG. 4A shows a top perspective view 400 of the device 100 of FIG. 1 for use on a left arm of the user according to an example embodiment, while FIG. 4B shows a top perspective view 450 of the device 100 of FIG. 1 for use on a right arm of the user according to an example embodiment. In alternate embodiments, the device 100 may be reversible such that a single device can be interchangeably used on both the right arm and the left arm. FIG. 5 shows a schematic diagram 500 illustrating a top perspective view of the device of FIGS. 4A and 4B secured on the left arm 502 and right arm 504 of the user respectively according to an example embodiment.

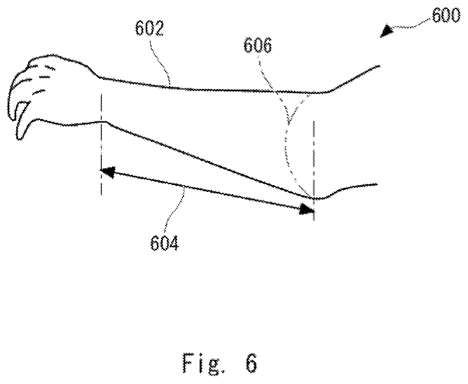

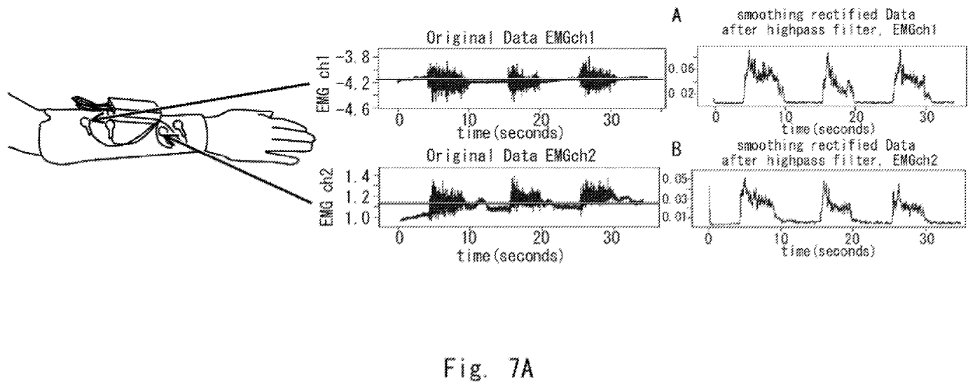

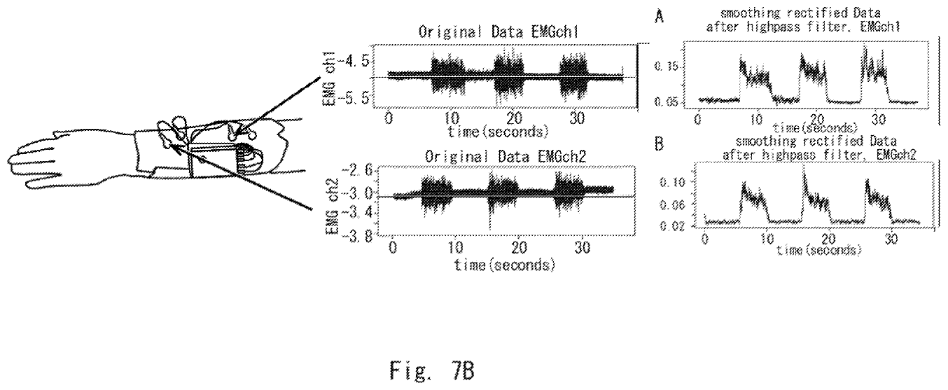

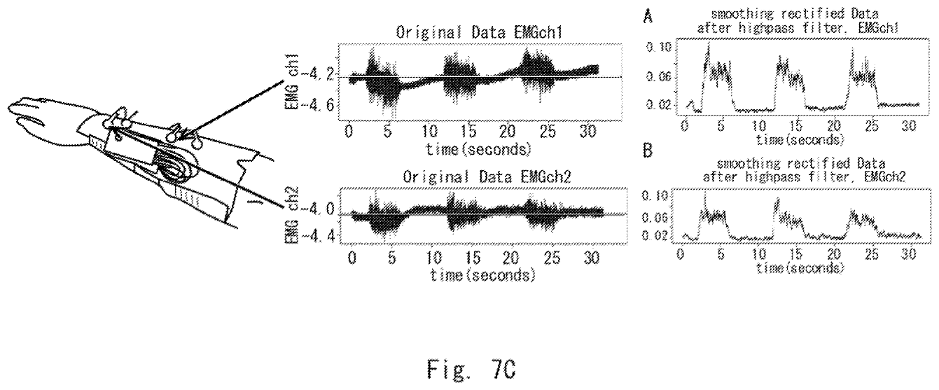

FIG. 6 shows a perspective view 600 illustrating dimensions of a forearm 602 of a user for which the device 100 of FIG. 1 is to be used in a study according to an example embodiment. In the following examples in FIGS. 7A to 7C, the device 100 was used on forearms 602 of different users having varying forearm lengths 604 and having varying arm circumferences 606. In these examples, the forearm length 604 is measured from the elbow axis to the Ulnar styloid, while the forearm circumference 606 is measured near the elbow. The following examples illustrate the use of the device 100 on different forearm dimensions with their respective data obtained from the forearm muscle groups.

FIGS. 7A to 7C show graphs illustrating forearm muscle group data of various users in the study using the device of FIG. 1, according to an example embodiment. In FIG. 7A, muscle group data is collected from a user having a forearm length of 25 centimeters (cm) and circumference of 20.5 centimeters (cm) using the device 100 and presented. In this case, the circumference of the user's forearm is smaller than an average circumference of a forearm. Muscle activity from the Extensor digitorum muscle group of the user is shown in Graph A of FIG. 7A, while muscle activity from the Extensor indicis muscle group of the user is shown in Graph B of FIG. 7A.

In FIG. 7B, muscle group data is collected and from a user having a forearm length of 30 centimeters (cm) and circumference of 26 centimeters (cm) using the device 100 and presented. In this case, the length of the user's forearm is longer than the average length of a forearm. Muscle activity from the Extensor digitorum muscle group of the user is shown in Graph A of FIG. 7B, while muscle activity from the Extensor indicis muscle group of the user is shown in Graph B of FIG. 7B.

In FIG. 7C, muscle group data is collected from a user having a forearm length of 25 centimeters (cm) and circumference of 26 centimeters (cm) using the device 100 and presented. In this case, the length of the user's forearm is shorter than the average length of a forearm while the circumference is larger than the average circumference of a forearm. Muscle activity from the Extensor digitorum muscle group of the user is shown in Graph A of FIG. 7C, while muscle activity from the Extensor indicis muscle group of the user is shown in Graph B of FIG. 7C. EMG data collected from the users as shown in FIGS. 7A to 7C provides the advantage of enabling an EMG signal of good quality to be obtained regardless of the size and length of the forearm. The device also enables the time spent on preparing electrode placements to be reduced. For example, it only take less than thirty seconds to wrap the present device around a forearm as compared to the more than four minutes it takes to wrap a conventional device around a forearm, the exact length of this being highly dependent on the skill of the user in finding the correct electrodes positions on the muscle group.

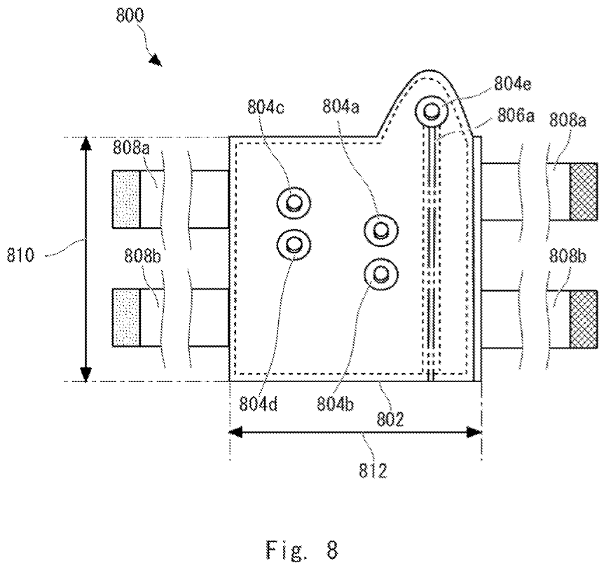

FIG. 8 shows a top perspective view of a device 800 for obtaining data of one or more muscle groups of a body for rehabilitation according to an example embodiment. The device 800 is similar to the device 100 of FIG. 1 and includes a support member 802, two or more electrodes 804a, 804b, 804c, 804d, 804e positioned on the support member 802 and one or more location identifiers 806a that are also positioned on the support member 802. The support member 802 may be elastic such that it can be stretched to adapt to parts of the body of a user having different sizes. Being stretchable, the support member 802 may be adapted to circumferentially enclose a portion of the body relating to the one or more muscle groups regardless of the body size. In addition, the support member 802 may be made from a soft, non-rigid material such as polyester or vinyl. The support member 802 may be dimensioned approximately 13 centimeters in length 810 and approximately 17 centimeters in width 812.

The two or more electrodes 804a, 804b, 804c, 804d, 804e may be configured to obtain data corresponding to the one or more muscle groups, while the one or more location identifiers 806a can be permanently fixed or removably attached to the support member 802. Even though five electrodes and one location identifier are shown in FIG. 8, it can be appreciated that other embodiments of the device 800 may include more than five electrodes and more than one location identifier. Each of the two or more electrodes 804a, 804b, 804c, 804d, 804e may be a disposable electrode such as a “peel-and-stick” electrode typically used in an electrocardiogram. Other types of electrodes, such as dry electrodes, surface electrodes, gelled electrodes, needle electrodes and/or fine wire electrodes are also possible. The two or more electrodes 804a, 804b, 804c, 804d, 804e may obtain data from the one or more muscle groups to detect medical abnormalities, an activation level of the muscle, or to analyze the biomechanics of the user.

In the embodiment where the two or more electrodes 804a, 804b, 804c, 804d, 804e may be removable from the support member 802, the device 800 may include two or more electrode mounts configured to receive the two or more electrodes 804a, 804b, 804c, 804d, 804e respectively. The function of the electrode mounts is to better secure the electrodes. In an embodiment, the two or more electrodes 804a, 804b, 804c, 804d, 804e may obtain electrical signals from the one or more muscle groups to assist in muscle activity. In an alternate embodiment, the two or more electrodes 804a, 804b, 804c, 804d, 804e may be configured to transmit data to the one or more muscle groups of the body. For example, the two or more electrodes 804a, 804b, 804c, 804d, 804e may transmit electrical signals to the one or more muscle groups to assist in muscle stimulation. The transmission of electrical signals for muscle stimulation may assist to relieve pain for the one or more muscle groups.

The device 800 may also include fastening means 808a, 808b so that the support member 802 can be removably secured against the body. The fastening means includes but is not limited to: a hook and loop fastener (e.g. a Velcro (®: Registered trademark) fastener), a buckle fastener and/or an adhesive fastener. It can be appreciated that other fastening means may be applicable such that the device 800 can be secured to and removed from the body with minimal effort.

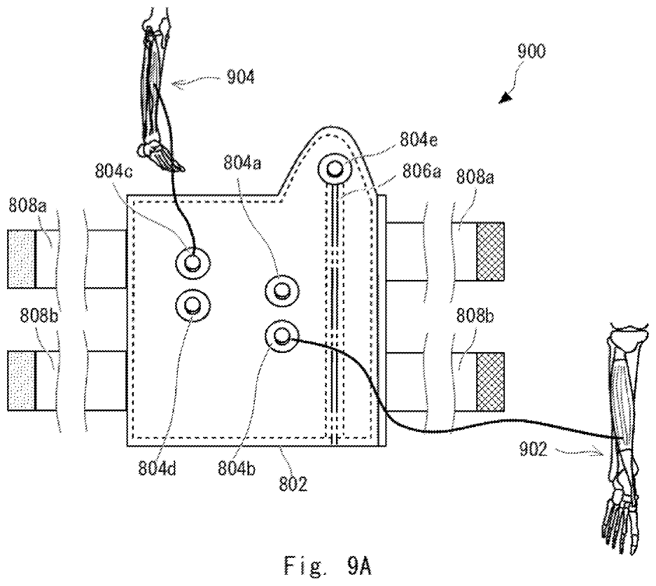

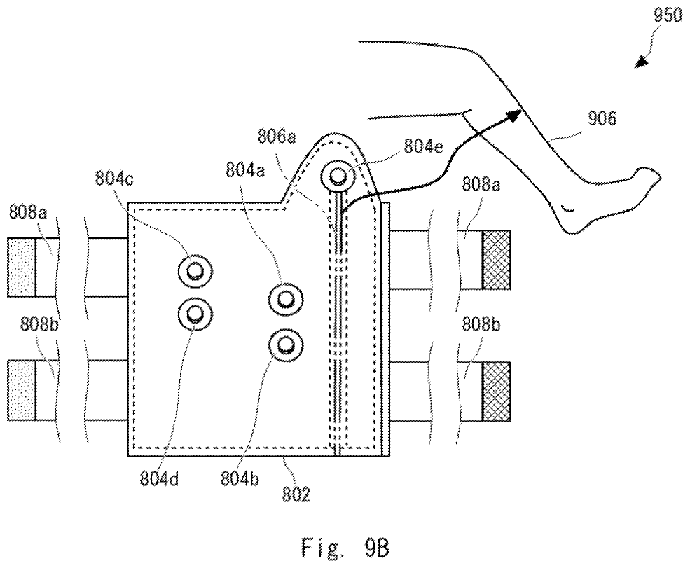

FIG. 9A shows a top perspective view 900 of the device 800 of FIG. 8 illustrating muscle groups of a lower limb in which data is obtained by the two or more electrodes 804a, 804b, 804c, 804d, 804e according to an example embodiment. In this embodiment, the one or more muscle groups of the body may refer to the muscle groups of a lower limb, such as a leg of the user. For example, and as shown in FIG. 9A, electrodes 804a and 804b may be positioned to obtain data corresponding to the Tibialis anterior muscle group 902 that is located in the lower limb of the user, while electrodes 804c and 804d may be positioned to obtain data corresponding to the Fibularis longus muscle group 904 that is also located in the lower limb of the user.

FIG. 9B shows a top perspective view 950 of the device 800 of FIG. 8 illustrating locations of the one or more location identifiers 806a according to an example embodiment. In this embodiment, the one or more location identifiers 806a may identify locations of a lower limb, i.e. a leg of the user. For example, and as shown in FIG. 9B, location identifier 806a may identify the location of the tibialis 906. When the device 800 is used on the leg of the user, the one or more location identifiers 806a identify respective locations such that one of the two or more electrodes 804a, 804b, 804c, 804d, 804e are positioned to obtain data corresponding to the one or more muscle groups of the leg.

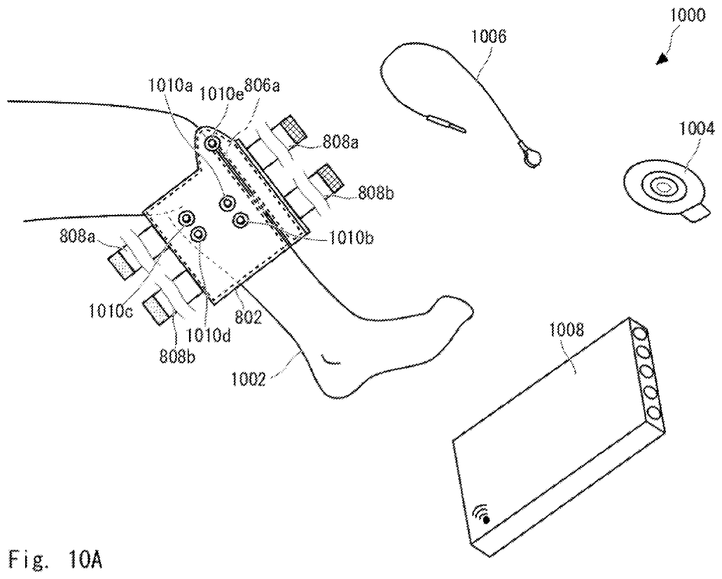

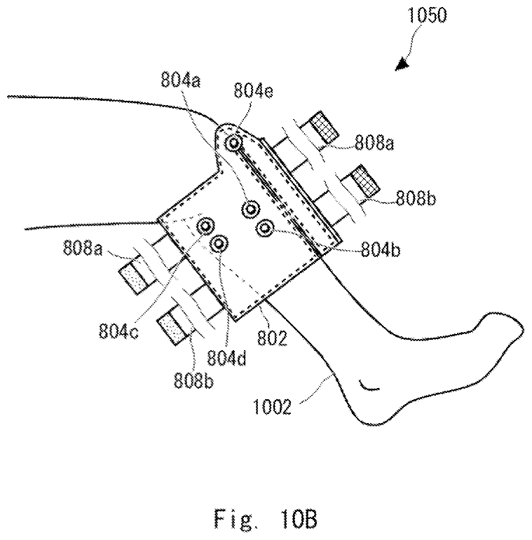

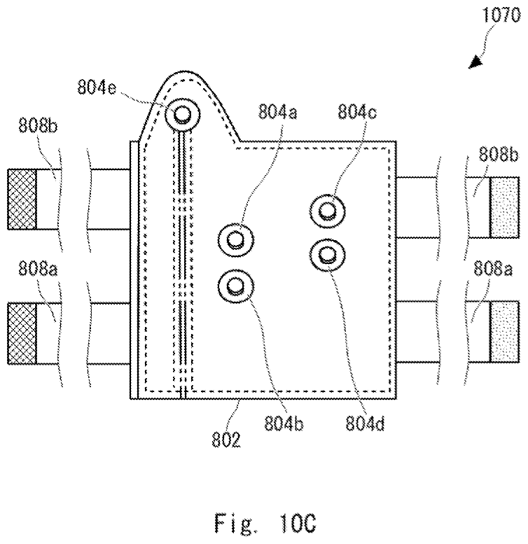

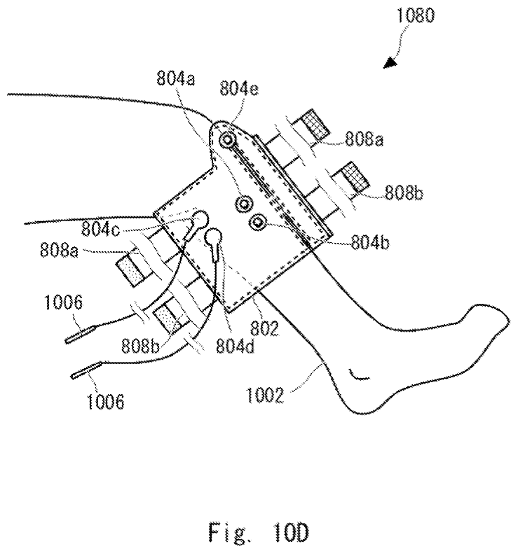

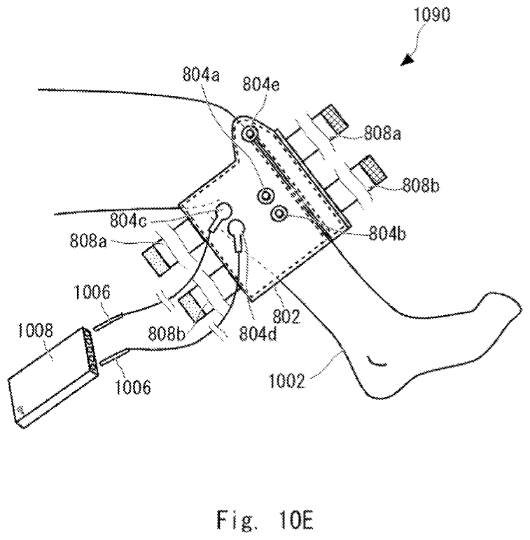

FIG. 10A shows a schematic diagram 1000 illustrating a top perspective view of the device 800 of FIG. 8 when used on a leg 1002 of a user according to an example embodiment. Before data is collected from the muscle groups, the device 800 is placed on the leg 1002 such that the support member 802 circumferentially encloses the leg 1002. The user may then adjust the device 800 such that the one or more location identifiers 806a is positioned at the respective locations as described previously. That is, location identifier 806a is positioned at the tibialis 906. In this way, electrodes 804a and 804b are positioned to obtain data corresponding to the Tibialis anterior muscle group 902 and electrodes 804c and 804d are positioned to obtain data corresponding to the Fibularis longus muscle group 904 and the Tibialis anterior muscle group 902. The device 800 is then secured to the leg 1002 by using the fastening means 808a, 808b.

In addition, each of the two or more electrodes 804a, 804b, 804c, 804d, 804e may be a disposable electrode 1004 that can be readily removed from and attached to the device 800. In this embodiment, the support member 802 includes two or more electrode mounts 1010a, 1010b, 1010c, 1010d, 1010e configured to receive the two or more electrodes 804a, 804b, 804c, 804d, 804e, respectively. The disposable electrode 1004 may be a typical “peel-and-stick” electrode that is only used once for hygiene purposes. The disposable electrode 1004 may also be attached to a snap cable 1006 to allow transmission of data to a wireless EMG sensor 1008. It can be appreciated that other types of electrodes, such as dry electrodes, surface electrodes, gelled electrodes, needle electrodes and/or fine wire electrodes are possible for each of the two or more electrodes 804a, 804b, 804c, 804d, 804e.

FIG. 10B shows a schematic diagram 1050 illustrating a top perspective view of the device 800 of FIG. 8 on the leg 1002 of the user with two or more disposable electrodes 804a, 804b, 804c, 804d, 804e attached thereto according to an example embodiment. After the device 800 is secured to the leg 1002, the two or more disposable electrodes 804a, 804b, 804c, 804d, 804e are attached to the device 800 via the two or more electrode mounts 1010a, 1010b, 1010c, 1010d, 1010e. Alternatively, the two or more disposable electrodes 804a, 804b, 804c, 804d, 804e may be attached to the device 800 before being secured to the leg 1002. In yet another alternative embodiment, the two or more disposable electrodes 804a, 804b, 804c, 804d, 804e may be permanently attached to the device 800 and therefore the device 800 is secured to the leg 1002 without the step of attaching the two or more disposable electrodes 804a, 804b, 804c, 804d, 804e. FIG. 10C shows a schematic diagram 1070 illustrating a bottom perspective view of the device 800 of FIG. 8 with two or more disposable electrodes 804a, 804b, 804c, 804d, 804e attached thereto according to an example embodiment. The functions of 804a, 804b, 804c, 804d, 804e in the bottom perspective view may have the same functions as illustrated in FIG. 10B.

FIG. 10D shows a schematic diagram 1080 illustrating a top perspective view of the device 800 of FIG. 8 on the leg 1002 of the user with two or more disposable electrodes 804a, 804b, 804c, 804d, 804e each having a snap cable 1006 attached thereto according to an example embodiment. After the two or more disposable electrodes 804a, 804b, 804c, 804d, 804e are mounted to the device 800, the snap cable 1006 may be attached to each of the two or more disposable electrodes 804a, 804b, 804c, 804d, 804e. In FIG. 10D, the electrodes 804c and 804d are each attached to a snap cable 1006 to facilitate transmission of data of the muscle groups. Each snap cable 1006 may be flexible with a “snap-on” feature that can be readily and securely attached to the electrode to provide strength and stability during the transmission of data.

FIG. 10E shows a schematic diagram 1090 illustrating a top perspective view of the device 800 of FIG. 8 on the leg 1002 of the user with two or more disposable electrodes 804a, 804b, 804c, 804d, 804e attached to a wireless EMG sensor 1008 according to an example embodiment. After attaching the snap cable 1006 to each of the two or more disposable electrodes 804a, 804b, 804c, 804d, 804e, each of the snap cables 1006 is subsequently connected to the wireless EMG sensor 1008. The wireless EMG sensor 1008 may be one that measures and records activity of the muscle groups from the data transmitted by the two or more disposable electrodes 804a, 804b, 804c, 804d, 804e. The wireless EMG sensor 1008 may be a portable sensor that can be worn by the user and can be used to analyze and assess the biomechanics of the user through muscle contractions, nerve conduction, muscular response in injured tissue and/or activation level. The data received by the wireless EMG sensor 1008 can also be transmitted wirelessly to a computing device for further analysis using a suitable software program. In FIG. 10E, the wireless EMG sensor 1008 is configured to receive data from at least one of the five electrodes. It can be appreciated that the wireless EMG sensor 1008 may be configured to receive data from more than five electrodes.

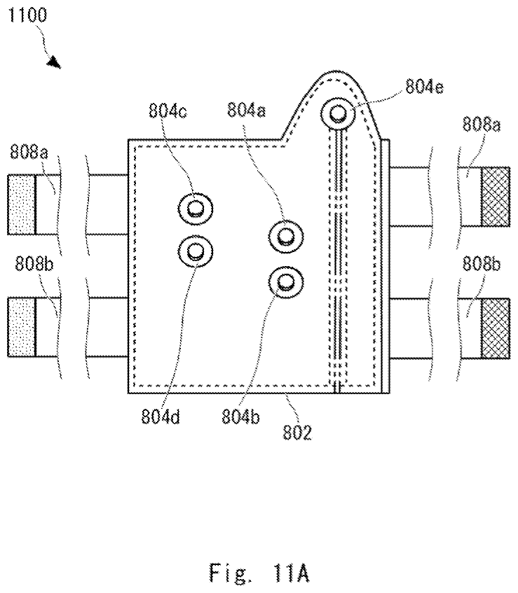

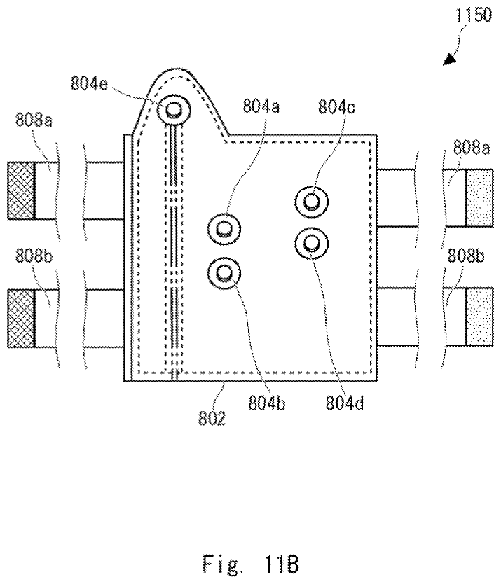

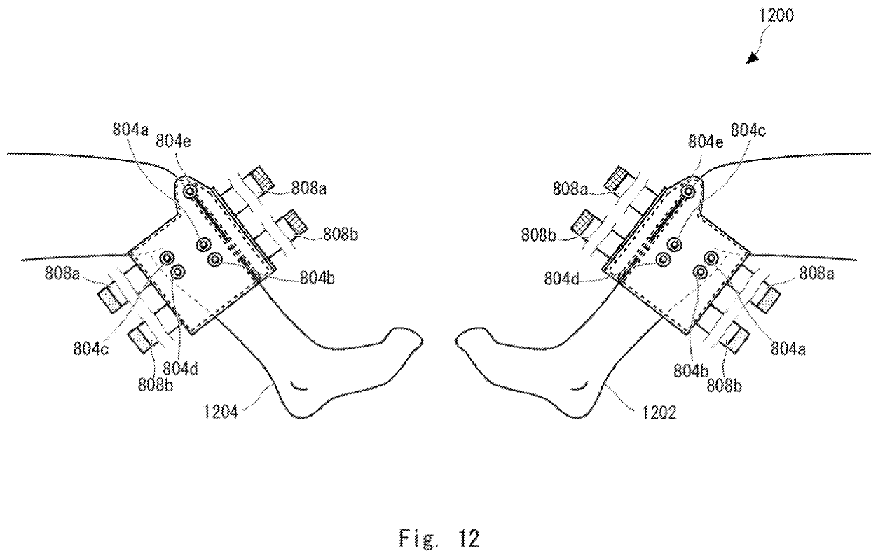

FIG. 11A shows a top perspective view 1100 of the device 800 of FIG. 8 for use on a right leg of the user according to an example embodiment, while FIG. 11B shows a top perspective view 1150 of the device 800 of FIG. 8 for use on a left leg of the user according to an example embodiment. In an alternate embodiment, the device 800 may be reversible such that a single device can be interchangeably used on both the right leg and the left leg. FIG. 12 shows a schematic diagram 1200 illustrating a top perspective view of the device of FIGS. 11A and 11B secured on the left leg 1202 and right leg 1204 of the user respectively according to an example embodiment.

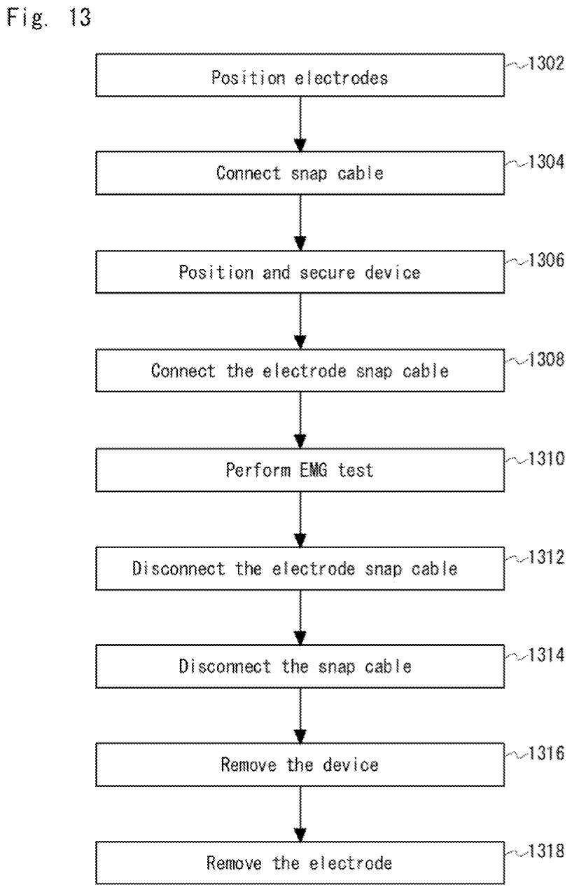

FIG. 13 shows a flow chart illustrating a method for obtaining data of one or more muscle groups of a body for rehabilitation according to an example embodiment. At step 1302, the two or more electrodes are positioned respectively on the two or more electrode mounts of the device. This can be achieved by placing an electrode snap button into a hole of the electrode mount. At step 1304, each of the two or more electrodes is connected to a snap cable. At step 1306, the device is positioned and secured on the body. The device is positioned such that each of the location identifiers of the device identifies a corresponding location of the body. The device is subsequently secured to the body using fastening means. Alternatively, the device may be positioned and secured to the body before the electrodes are attached to the device. In further embodiments in which the electrodes are permanently fixed onto the device, step 1302 may be omitted.

At step 1308, each of the electrode snap cables is connected to an EMG sensor. The EMG sensor may be a wireless sensor that transmits data wirelessly to a computing device via Bluetooth (®: Registered trademark) or Wi-Fi (®: Registered trademark). At step 1310, an EMG test is performed to obtain data of one or more muscle groups of the body. After the EMG test is completed, each of the electrode snap cables is disconnected from the EMG sensor at step 1312. At step 1314, each of the snap cables is disconnected from the two or more electrodes. At step 1316, the device is removed from the body and at step 1318, the two or more electrodes are removed from the body part.

Embodiments of the present disclosure provide a device for obtaining data of one or more muscle groups of a body for rehabilitation that overcome the problems of previous devices. As described above, the example embodiments also provide a method for manufacturing the device which can be worn easily while consistently locating the correct position of the specific muscle group of the body, regardless of the body size or body weight. The device in embodiments of the present disclosure also automatically locates the specific muscle group of the body. The device may also be used by a user (or patient) outside of a clinic or a laboratory (for example, at home) as it can be readily secured to and removed from the body using the fastening means. Advantageously, this easy securing to and removing of the device from the body results in obtaining high quality and consistent data during a long use of the device, as prolonged usage may cause insufficient contact between the body and the electrode.

In addition, the device of the present disclosure reduces the problem of inaccurate placement of the EMG electrode on the muscle group. It also reduces noise level in the signal (or data) and is portable so that the user (or patient) is able to use it at home for rehabilitation or tele-rehabilitation. It can be appreciated that the device in embodiments of the present disclosure can be used for body parts other than an upper limb or a lower limb and can be used on either side of the limbs. The device can also be used repeatedly and consistently maintain contact between the body and the electrode. By having disposable and washable electrodes, the device can be used by different users and yet is still hygienic. Further, the use of disposable electrodes also reduces the cost for a user and the device may also reduce the time needed by an EMG examiner to conduct muscle checking, attach the electrodes and analyze the muscle data. These effects can be achieved by initiating an EMG sensor application when the EMG wireless sensor transmits the data to the EMG examiner even when the device is used at home.

### Second Example Embodiment

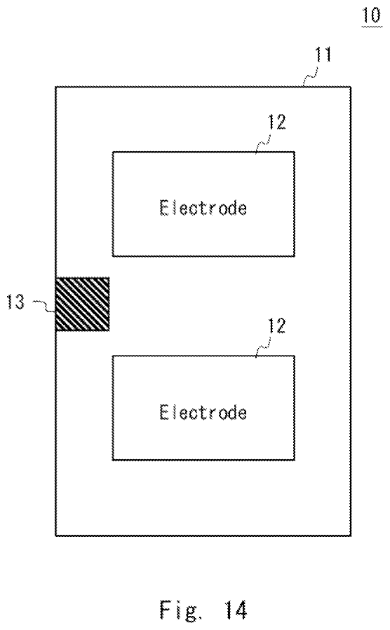

A Second example embodiment of the disclosure is explained below referring to FIG. 14. The second example embodiment shows generic concept of this disclosure; however, it does not show the limit of this disclosure.

FIG. 14 shows a block diagram of a device 10. The apparatus 10 includes a support member 11, two electrodes 12 and one location identifier 13. However, there may be more electrodes 12 of the device 10 in this example embodiment. Similarly, there may be a plurality of location identifiers 13 of the device 10 in this example embodiment.

The electrodes 12 are configured to obtain data and positioned on the support member 11. The location identifier 13 positioned on the support member 11 and the location identifier 13 identifies a location to position one of the two electrodes 12 to obtain data corresponding to one or more muscle groups of a body.

Furthermore, a method of manufacturing the device 10 can be explained as follows:

providing the support member 11;

positioning the two electrodes 12 on the support member 11, the electrodes 12 configured to obtain data; and

positioning the one location identifier 13 on the support member 11, the location identifier 13 identifying a location to position one of the electrodes 12 to obtain data corresponding to one or more muscle groups of a body.

The location identifier 13 identifies a location to position one of the two electrodes 12 to obtain data corresponding to one or more muscle groups of a body, therefore, the device 10 can obtain data of one or more muscle groups of a body for rehabilitation. The method shown above can manufacture this device.

Part of or all the foregoing embodiments can be described as in the following appendixes, but the present disclosure is not limited thereto.

### (Supplementary Note 1)

A device comprising:

a support member;

two or more electrodes configured to obtain data, the two or more electrodes being positioned on the support member; and

one or more location identifiers positioned on the support member, each of the one or more location identifiers identifying a location to position one of the two or more electrodes to obtain data corresponding to one or more muscle groups of a body.

### (Supplementary Note 2)

The device according to Supplementary Note 1, further comprising two or more electrodes mount configured to receive the two or more electrodes, respectively.

### (Supplementary Note 3)

The device according to Supplementary Note 1 or 2, wherein the support member is adapted to circumferentially enclose a portion of the body, the portion of the body relating to the one or more muscle groups.

### (Supplementary Note 4)

The device according to any one of Supplementary Notes 1 to 3, further comprising fastening means for removably securing the support member against the body.

### (Supplementary Note 5)

The device according to Supplementary Note 4, wherein the fastening means comprises a hook and loop fastener.

### (Supplementary Note 6)

The device according to any one of Supplementary Notes 1 to 5, wherein the two or more electrodes are further configured to transmit data to the one or more muscle groups of the body.

### (Supplementary Note 7)

The device according to any one of Supplementary Notes 1 to 6, wherein the one or more muscle groups of a body comprises at least: one or more muscle groups of an upper limb or one or more muscle groups of a lower limb.

### (Supplementary Note 8)

The device according to any one of Supplementary Notes 1 to 7, wherein each of the one or more location identifiers identifying the location comprises at least one of: an ulnar styloid, an elbow joint width, an elbow axis, an olecranon or a tibialis.

### (Supplementary Note 9)

The device according to any one of Supplementary Notes 1 to 8, wherein the two or more electrodes comprise disposable electrodes.

### (Supplementary Note 10)

A method of manufacturing a device, the method comprising:

providing a support member;

positioning two or more electrodes on the support member, the two or more electrodes configured to obtain data; and

positioning one or more location identifiers on the support member, each of the one or more location identifiers identifying a location to position one of the two or more electrodes to obtain data corresponding to one or more muscle groups of a body.

### (Supplementary Note 11)

The method according to Supplementary Note 10, further comprising providing two or more electrode mounts configured to receive the two or more electrodes, respectively.

### (Supplementary Note 12)

The method according to Supplementary Note 10 or 11, wherein the support member is adapted to circumferentially enclose a portion of the body, the portion of the body relating to the one or more muscle groups.

### (Supplementary Note 13)

The method according to any one of Supplementary Notes 10 to 12, further comprising providing fastening means for removably securing the support member against the body.

### (Supplementary Note 14)

The method according to Supplementary Note 13, wherein the fastening means comprises a hook and loop fastener.

### (Supplementary Note 15)

The method according to any one of Supplementary Notes 10 to 14, wherein the two or more electrodes are further configured to transmit data to the one or more muscle groups of the body.

### (Supplementary Note 16)

The method according to any one of Supplementary Notes 10 to 15, wherein the one or more muscle groups of a body comprises at least: one or more muscle groups of an upper limb or one or more muscle groups of a lower limb.

### (Supplementary Note 17)

The method according to any one of Supplementary Notes 10 to 16, wherein each of the one or more location identifiers identifying the location comprises at least one of: an ulnar styloid, an elbow joint width, an elbow axis, an olecranon or a tibialis.

### (Supplementary Note 18)

The method of according to any one of Supplementary Notes 10 to 17, wherein the two or more electrodes comprise disposable electrodes.

It will be appreciated by a person skilled in the art that numerous variations and/or modifications may be made to the present disclosure as shown in the specific embodiments without departing from the spirit or scope of this disclosure as broadly described. The present embodiments are, therefore, to be considered in all respects to be illustrative and not restrictive.

This application is based upon and claims the benefit of priority from Singapore patent application No. 10201906405P, filed on Jul. 10, 2019, the disclosure of which is incorporated herein in its entirety by reference.

### REFERENCE SIGNS LIST

10 device
11 support member
12 electrode
13 location identifier
100, 800 device
102, 802 support member
104, 804 electrode
106, 806 location identifier
108, 808 fastening means
304, 1004 disposable electrode
306, 1006 snap cable
308, 1008 wireless EMG sensor
310, 1010 electrode mount

What is claimed is:

1. A device comprising:
a support member;
two or more electrodes configured to obtain data, the two or more electrodes being positioned on the support member; and
one or more location identifiers positioned on the support member, each of the one or more location identifiers identifying a location to position one of the two or more electrodes to obtain data corresponding to one or more muscle groups of a body.

2. The device according to claim 1, further comprising two or more electrode mounts configured to receive the two or more electrodes, respectively.

3. The device according to claim 1, wherein the support member is adapted to circumferentially enclose a portion of the body, the portion of the body relating to the one or more muscle groups.

4. The device according to claim 1, further comprising fastening means for removably securing the support member against the body.

5. The device according to claim 4, wherein the fastening means comprises a hook and loop fastener.

6. The device according to claim 1, wherein the two or more electrodes are further configured to transmit data to the one or more muscle groups of the body.

7. The device according to claim 1, wherein the one or more muscle groups of a body comprises at least: one or more muscle groups of an upper limb or one or more muscle groups of a lower limb.

8. The device according to claim 1, wherein each of the one or more location identifiers identifying the location comprises at least one of: an ulnar styloid, an elbow joint width, an elbow axis, an olecranon or a tibialis.

9. The device according to claim 1, wherein the two or more electrodes comprise disposable electrodes.

10. A method of manufacturing a device, the method comprising:
providing a support member;
positioning two or more electrodes on the support member, the two or more electrodes configured to obtain data; and
positioning one or more location identifiers on the support member, each of the one or more location identifiers identifying a location to position one of the two or more electrodes to obtain data corresponding to one or more muscle groups of a body.

11. The method according to claim 10, further comprising providing two or more electrode mounts configured to receive the two or more electrodes, respectively.

12. The method according to claim 10, wherein the support member is adapted to circumferentially enclose a portion of the body, the portion of the body relating to the one or more muscle groups.

13. The method according to claim 10, further comprising providing fastening means for removably securing the support member against the body.

14. The method according to claim 13, wherein the fastening means comprises a hook and loop fastener.

15. The method according to claim 10, wherein the two or more electrodes are further configured to transmit data to the one or more muscle groups of the body.

16. The method according to claim 10, wherein the one or more muscle groups of a body comprises at least: one or more muscle groups of an upper limb or one or more muscle groups of a lower limb.

17. The method according to claim 10, wherein each of the one or more location identifiers identifying the location comprises at least one of: an ulnar styloid, an elbow joint width, an elbow axis, an olecranon or a tibialis.

18. The method of according to claim 10, wherein the two or more electrodes comprise disposable electrodes.
